(19) United States
(12) Patent Application Publication
(10) Pub. No.: US 2024/0046520 A1
(43) Pub. Date: Feb. 8, 2024

(54) SYSTEMS AND METHODS FOR CALIBRATING IMAGE ACQUISITION DEVICES

(71) Applicant: SHANGHAI UNITED IMAGING HEALTHCARE CO., LTD., Shanghai (CN)

(72) Inventors: Qinhua HUANG, Shanghai (CN); Jianqiao CHEN, Shanghai (CN); Shiming HU, Shanghai (CN)

(73) Assignee: SHANGHAI UNITED IMAGING HEALTHCARE CO., LTD., Shanghai (CN)

(21) Appl. No.: 18/491,146

(22) Filed: Oct. 20, 2023

Related U.S. Application Data
(63) Continuation of application No. PCT/CN2022/088020, filed on Apr. 20, 2022.

(30) Foreign Application Priority Data
Apr. 20, 2021 (CN) 202110423744.4

(51) Int. Cl. G06T 7/80; A61B 5/00; G06T 7/60

(52) U.S. Cl. CPC G06T 7/80; A61B 5/704; G06T 7/60; G06T 2207/30204; G06T 2207/10072

(57) ABSTRACT

A system and method for calibrating an image acquisition device are provided. First characteristic information of one or more optical markers in a first coordinate system applied to a medical device may be obtained. Multiple images each of which includes the one or more optical markers acquired by an image acquisition device may be obtained. Second characteristic information of the one or more optical markers in a second coordinate system applied to the multiple images acquired by the image acquisition device based on the multiple images may be obtained. A transform relationship between the first coordinate system and the second coordinate system may be determined based on the first characteristic information and the second characteristic information of the one or more optical markers.

### CROSS REFERENCE TO RELATED APPLICATION

This application is a continuation of International Application No. PCT/CN2022/088020, filed on Apr. 20, 2022, which claims priority to Chinese Patent Application No. 202110423744.4, filed on Apr. 20, 2021, the contents of which are hereby incorporated by reference.

### TECHNICAL FIELD

The present disclosure relates to calibrations of medical scan systems, and in particular, to systems and methods for calibrating image acquisition devices of medical systems.

### BACKGROUND

Image acquisition devices (e.g., a camera) have been widely used in medical systems. It is necessary to calibrate parameters of the image acquisition device, which is vital for the accuracy of output results of the image acquisition device.

Thus, it is desired to provide systems and methods for calibrating an image acquisition device of a medical system with high efficiency and accuracy.

### SUMMARY

According to an aspect of the present disclosure, a system for calibrating an image acquisition device may be provided. The system may include at least one storage device including a set of instructions and at least one processor configured to communicate with the at least one storage device. When executing the set of instructions, the at least one processor may be directed to cause the system to perform one or more of the following operations. The system may obtain first characteristic information of one or more optical markers in a first coordinate system applied to a medical device. The system may also obtain multiple images each of which includes the one or more optical markers acquired by an image acquisition device. A field of view of the image acquisition device may cover a scan region of the medical device. The system may obtain second characteristic information of the one or more optical markers in a second coordinate system applied to the multiple images acquired by the image acquisition device based on the multiple images. The system may further determine a transform relationship between the first coordinate system and the second coordinate system based on the first characteristic information and the second characteristic information of the one or more optical markers.

In some embodiments, the one or more optical markers may be disposed on at least one of a couch of the medical device or an inner wall of a bore of the medical device.

In some embodiments, at least one of the one or more optical markers may be disposed on an end of a couch of the medical device near a bore of the medical device.

In some embodiments, an optical marker among the one or more optical markers may be disposed on a couch of the medical device, the first characteristic information of the optical marker may include first position information of the optical marker, and to obtain the first characteristic information of the optical marker in the first coordinate system applied to the medical device, the system may obtain positions of the couch in the first coordinate system during movement of the couch. The system may further determine the first position information of the optical marker in the first coordinate system based on the positions of the couch in the first coordinate system.

In some embodiments, the at least one processor may be further directed to cause the system to perform one or more of the following operations. The system may obtain multiple groups of status parameters of a couch of the medical device. Each group may include a load and a first extension of the couch along a longitudinal direction of the couch. For each group of the multiple groups of status parameters of the couch, the system may perform one or more of the following operations. The system may obtain a first image of the couch including an optical marker disposed on the couch acquired by the image acquisition device when the couch is under the load and the first extension in the group. The system may determine a first position of the optical marker in the second coordinate system based on the first image of the couch. The system may also determine a first deformation level of the couch in the first coordinate system based on the first position of the optical marker and a prior transform relationship between the first coordinate system and the second coordinate system. The system may further perform a calibration on one or more performance parameters of the couch to obtain a calibration result based on the multiple groups of the status parameters of the couch and first deformation levels corresponding to the multiple groups of the status parameters of the couch.

In some embodiments, the at least one processor may be further directed to cause the system to perform one or more of the following operations. The system may obtain a weight of an object and a second extension of a couch of the medical device after the couch moves the object to a position in a bore of the medical device. The system may also obtain a second image of the couch including the optical marker disposed on the couch acquired by the image acquisition device after the couch moves the object to the position in the bore of the medical device. The system may also determine a second position of the optical marker in the second coordinate system based on the second image. The system may also determine a second deformation level of the couch in the first coordinate system based on the second position of the optical marker and the transform relationship between the first coordinate system and the second coordinate system. The system may further determine an assessment result of the couch based on the weight of the object, the second extension, the second deformation level of the couch, and a calibration result corresponding to one or more performance parameters of the couch.

In some embodiments, the at least one processor may be further directed to cause the system to perform one or more of the following operations. The system may obtain a first image and a second image each of which includes a surface of an object being scanned acquired by the image acquisition device. The system may also obtain a first position of the surface in the second coordinate system based on the first image and a second position of the surface in the second coordinate system based on the second image. The system may determine motion information of the surface of the object in the first coordinate system based on the first position and the second position of the surface of the object and the transform relationship between the first coordinate system and the second coordinate system.

In some embodiments, the at least one processor may be further directed to cause the system to perform one or more of the following operations. The system may obtain an image of the one or more optical markers acquired by the image acquisition device. The one or more optical markers may have different distances from the image acquisition device. The system may also determine sizes of the one or more optical markers in the image based on sizes of the one or more optical markers in the first coordinate system, and the transform relationship. The system may determine resolutions of the image acquisition device for different distances from the image acquisition device based on the sizes of the one or more optical markers in the image.

According to an aspect of the present disclosure, a system for determining an assessment result of a couch of a medical device may be provided. The system may include at least one storage device including a set of instructions and at least one processor configured to communicate with the at least one storage device. When executing the set of instructions, the at least one processor may be directed to cause the system to perform one or more of the following operations. The system may obtain a weight of an object and an extension of a couch of a medical device along a longitudinal direction of the couch after the couch moves the object to a position in a bore of the medical device. The system may obtain an image of the couch including an optical marker disposed on the couch acquired by an image acquisition device after the couch moves the object to the position in the bore of the medical device. A field of view of the image acquisition device may cover a scan region of the medical device. The system may obtain a transform relationship between a first coordinate system applied to the medical device and a second coordinate system applied to the image acquisition device. The system may also determine a position of the optical marker in the second coordinate system based on the image of the couch. The system may also determine a deformation level of the couch in the first coordinate system based on the position of the optical marker and the transform relationship between the first coordinate system and the second coordinate system. The system may determine an assessment result of the couch based on the weight of the object, the extension, the deformation level of the couch, and a calibration result corresponding to one or more performance parameters of the couch.

According to yet another aspect of the present disclosure, a method for calibrating an image acquisition device may be provided. The method may include obtaining first characteristic information of one or more optical markers in a first coordinate system applied to a medical device. The method may also include obtaining multiple images each of which includes the one or more optical markers acquired by an image acquisition device. A field of view of the image acquisition device may cover a scan region of the medical device. The method may also include obtaining second characteristic information of the one or more optical markers in a second coordinate system applied to the multiple images acquired by the image acquisition device based on the multiple images. The method may further include determining a transform relationship between the first coordinate system and the second coordinate system based on the first characteristic information and the second characteristic information of the one or more optical markers.

According to yet another aspect of the present disclosure, a method for determining an assessment result of a couch of a medical device may be provided. The method may include obtaining a weight of an object and an extension of a couch of a medical device along a longitudinal direction of the couch after the couch moves the object to a position in a bore of the medical device. The method may include obtaining an image of the couch including an optical marker disposed on the couch acquired by an image acquisition device after the couch moves the object to the position in the bore of the medical device. A field of view of the image acquisition device may cover a scan region of the medical device. The method may also include obtaining a transform relationship between a first coordinate system applied to the medical device and a second coordinate system applied to the image acquisition device. The method may also include determining a position of the optical marker in the second coordinate system based on the image of the couch. The method may also include determining a deformation level of the couch in the first coordinate system based on the position of the optical marker and the transform relationship between the first coordinate system and the second coordinate system. The method may further include determining an assessment result of the couch based on the weight of the object, the extension, the deformation level of the couch, and a calibration result corresponding to one or more performance parameters of the couch.

According to yet another aspect of the present disclosure, a non-transitory computer readable medium may be provided. The non-transitory computer readable may include at least one set of instructions for calibrating an image acquisition device. When executed by at least one processor of a computing device, the at least one set of instructions may cause the computing device to perform a method. The method may include obtaining first characteristic information of one or more optical markers in a first coordinate system applied to a medical device. The method may also include obtaining multiple images each of which includes the one or more optical markers acquired by an image acquisition device. A field of view of the image acquisition device may cover a scan region of the medical device. The method may also include obtaining second characteristic information of the one or more optical markers in a second coordinate system applied to the multiple images acquired by the image acquisition device based on the multiple images. The method may further include determining a transform relationship between the first coordinate system and the second coordinate system based on the first characteristic information and the second characteristic information of the one or more optical markers.

According to yet another aspect of the present disclosure, a non-transitory computer readable medium may be provided. The non-transitory computer readable may include at least one set of instructions for determining an assessment result of a couch of a medical device. When executed by at least one processor of a computing device, the at least one set of instructions may cause the computing device to perform a method. The method may include obtaining a weight of an object and an extension of a couch of a medical device along a longitudinal direction of the couch after the couch moves the object to a position in a bore of the medical device. The method may include obtaining an image of the couch including an optical marker disposed on the couch acquired by an image acquisition device after the couch moves the object to the position in the bore of the medical device. A field of view of the image acquisition device may cover a scan region of the medical device. The method may also include obtaining a transform relationship between a first coordinate system applied to the medical device and a second coordinate system applied to the image acquisition device. The method may also include determining a position of the optical marker in the second coordinate system based on the image of the couch. The method may also include determining a deformation level of the couch in the first coordinate system based on the position of the optical marker and the transform relationship between the first coordinate system and the second coordinate system. The method may further include determining an assessment result of the couch based on the weight of the object, the extension, the deformation level of the couch, and a calibration result corresponding to one or more performance parameters of the couch.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a schematic diagram illustrating hardware and/or software components of an exemplary computing device on which the processing device 120 may be implemented according to some embodiments of the present disclosure;

FIG. 3 is a schematic diagram illustrating hardware and/or software components of an exemplary mobile device according to some embodiments of the present disclosure;

FIG. 4 is a block diagram illustrating an exemplary processing device according to some embodiments of the present disclosure;

FIG. 5A is a flowchart illustrating an exemplary process for calibrating an image acquisition device of a medical system according to some embodiments of the present disclosure;

FIG. 5B is a schematic diagram illustrating an exemplary optical marker according to some embodiments of the present disclosure;

FIG. 6 is a flowchart illustrating an exemplary process for calibrating one or more performance parameters of a couch of a medical device according to some embodiments of the present disclosure;

FIG. 7 is a side view of the medical device 110 in FIG. 1 according to some embodiments of the present disclosure;

FIG. 8 is a flowchart illustrating an exemplary process 800 for determining an assessment result of a couch of a medical device according to some embodiments of the present disclosure;

FIG. 9 is a flowchart illustrating an exemplary process 900 for determining motion information of a surface of an object being scanned in a first coordinate system applied to a medical device according to some embodiments of the present disclosure; and

FIG. 10 is a flowchart illustrating an exemplary process 1000 for determining resolutions of an image acquisition device of a medical system according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “pixel” and “voxel” in the present disclosure are used interchangeably to refer to an element of an image. An anatomical structure shown in an image of a subject may correspond to an actual anatomical structure existing in or on the subject's body.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

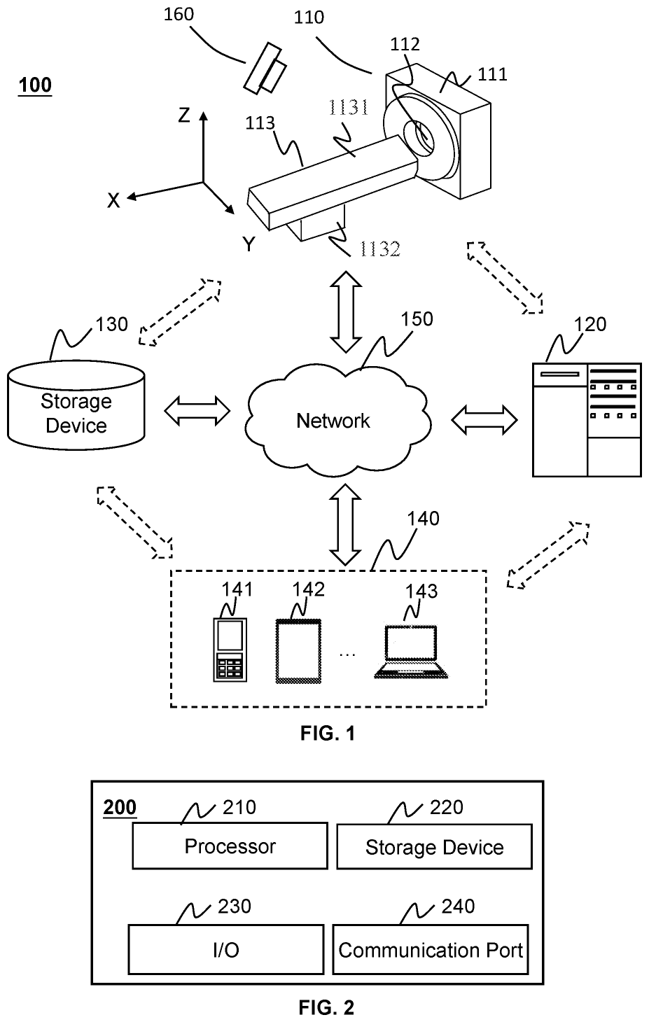

FIG. 1 is a schematic diagram illustrating an exemplary medical system 100 according to some embodiments of the present disclosure. As shown in FIG. 1, the medical system 100 may include a medical device 110, a processing device 120, a storage device 130, one or more terminals 140, a network 150, and an image acquisition device 160. In some embodiments, the medical device 110, the processing device 120, the storage device 130, the terminal(s) 140, and/or the image acquisition device 160 may be connected to and/or communicate with each other via a wireless connection, a wired connection, or a combination thereof. The connection between the components of the medical system 100 may be variable. Merely by way of example, the medical device 110 may be connected to the processing device 120 through the network 150 or directly. As a further example, the storage device 130 may be connected to the processing device 120 through the network 150 or directly.

The medical device 110 may be configured to scan and/or treat a subject. Merely by way of example, the medical device 110 may generate or provide image data related to a subject via scanning the subject. In some embodiments, the subject may include a biological subject and/or a non-biological subject. For example, the subject may include a specific portion of a body, such as the head, the thorax, the abdomen, or the like, or a combination thereof. As another example, the subject may be a man-made composition of organic and/or inorganic matters that are with or without life.

In some embodiments, the medical device 110 may be a single-modality apparatus. For example, the medical device 110 may include an imaging device. The imaging device may be configured to provide the imaging data for determining the at least one part of the subject (e.g., an anatomical point). The imaging device may include a CT device, a CBCT device, a PET device, a volume CT device, an MRI device, a SPECT device, or the like, or a combination thereof. In some embodiments, the medical device 110 may be a multi-modality (e.g., two-modality) apparatus. For example, the imaging device may include a CT-PET device, a CT-MRI device, a PET-MRI device, a SPECT-CT device, or the like, or a combination thereof. As another example, the medical device 110 may include an interventional medical device. Exemplary interventional medical devices may include a radiation therapy (RT) device, an ultrasound treatment device, a thermal treatment device, a surgical intervention device, or the like, or a combination thereof.

For example, the medical device may include a gantry 111, a bore 112, a couch 113 (also referred as to a scanning table 113). The gantry 111 may be configured to support one or more components of the medical device 110. The bore 112 may be used to accommodate a subject to be imaged and/or treated. The couch 113 may be configured to support and/or transfer the at least one part of the subject to, for example, a scanning region or a treatment region of the medical device 110. The couch 113 may include a table top 1131, a supporting component 1132, etc. The supporting component 1132 may be configured to support the table top 1131. The couch 113 may move in any direction. For example, a longitudinal direction (i.e., along a long axis of table top 1131 in the plane of the table top 1131), a lateral direction (i.e., along a short axis of the table top 1131 in the plane of the table top 1131), a direction (also referred to as a vertical direction) perpendicular to the longitudinal direction and lateral direction, or a direction oblique to the longitudinal direction and/or the lateral direction. The movement of the couch 113 may be driven manually or by, for example, a motor. In some embodiments, the longitudinal direction may be described as X direction as shown in FIG. 1. The lateral direction may be described as Y direction as shown in FIG. 1. The vertical direction may be described as the Z direction as shown in FIG. 1.

The processing device 120 may process data and/or information obtained from the medical device 110, the storage device 130, the terminal(s) 140, and/or the image acquisition device 160. For example, the processing device 120 may obtain first characteristic information of one or more optical markers in a first coordinate system applied to the medical device 110. The processing device 120 may also obtain multiple images each of which includes the one or more optical markers acquired by the image acquisition device 160. A field of view of the image acquisition device 160 may cover a scan region of the medical device 110. The processing device 120 may obtain second characteristic information of the one or more optical markers in a second coordinate system applied to the image acquisition device 160 based on the multiple images. The processing device 120 may further determine a transform relationship between the first coordinate system and the second coordinate system based on the first characteristic information and the second characteristic information of the one or more optical markers.

In some embodiments, the processing device 120 may be a single server or a server group. The server group may be centralized or distributed. In some embodiments, the processing device 120 may be local to or remote from the medical system 100. For example, the processing device 120 may access information and/or data from the medical device 110, the storage device 130, the terminal(s) 140, and/or the image acquisition device 160 via the network 150. As another example, the processing device 120 may be directly connected to the medical device 110, the terminal(s) 140, the storage device 130, and/or the image acquisition device 160 to access information and/or data. In some embodiments, the processing device 120 may be implemented on a cloud platform. In some embodiments, the processing device 120 may be implemented by a computing device 200 having one or more components as described in connection with FIG. 2. In some embodiments, the processing device 120 may include one or more processors (e.g., single-core processor(s) or multi-core processor(s)).

The storage device 130 may store data, instructions, and/or any other information. In some embodiments, the storage device 130 may store data obtained from the processing device 120, the terminal(s) 140, the medical device 110, and/or the image acquisition device 160. For example, the storage device 130 may store image data collected by the image acquisition device 160. As another example, the storage device 130 may store the transform relationship between the first coordinate system and the second coordinate system. In some embodiments, the storage device 130 may store data and/or instructions that the processing device 120 may execute or use to perform exemplary methods described in the present disclosure. In some embodiments, the storage device 130 may include a mass storage device, a removable storage device, a volatile read-and-write memory, a read-only memory (ROM), or the like, or any combination thereof.

In some embodiments, the storage device 130 may be connected to the network 150 to communicate with one or more other components of the medical system 100 (e.g., the processing device 120, the terminal(s) 140). One or more components of the medical system 100 may access the data or instructions stored in the storage device 130 via the network 150. In some embodiments, the storage device 130 may be part of the processing device 120.

The terminal(s) 140 may enable user interaction between a user and the medical system 100. In some embodiments, the terminal(s) 140 may include a mobile device 141, a tablet computer 142, a laptop computer 143, or the like, or any combination thereof. In some embodiments, the terminal(s) 140 may include an input device, an output device, etc. In some embodiments, the terminal(s) 140 may be part of the processing device 120.

The network 150 may include any suitable network that can facilitate the exchange of information and/or data for the medical system 100. In some embodiments, one or more components of the medical system 100 (e.g., the medical device 110, the processing device 120, the storage device 130, the terminal(s) 140) may communicate information and/or data with one or more other components of the medical system 100 via the network 150. The network 150 may be or include a public network (e.g., the Internet), a private network (e.g., a local area network (LAN)), a wired network, a wireless network (e.g., an 802.11 network, a Wi-Fi network), a frame relay network, a virtual private network (VPN), a satellite network, a telephone network, routers, hubs, switches, server computers, and/or any combination thereof. In some embodiments, the network 150 may include one or more network access points.

The image acquisition device 160 may be configured to capture image data of a bore of the medical device 110 (e.g., a scan region). For example, the image acquisition device 160 may be configured to capture image data of the subject before, during, and/or after the medical device 110 performs a scan or a treatment on the subject. As another example, the image acquisition device 160 may be configured to acquire depth image data of the subject. The depth image data may refer to image data that includes depth information of each physical point on the body surface of the subject, such as a distance from each physical point to a specific point (e.g., an optical center of the image acquisition device 160). The depth image data may be captured by a range sensing device, e.g., a structured light scanner, a time-of-flight (TOF) device, a stereo triangulation camera, a sheet of light triangulation device, an interferometry device, a coded aperture device, a stereo matching device, or the like, or any combination thereof. As yet another example, the image acquisition device 160 may be configured to capture image data of one or more optical markers disposed on the couch 113 of the medical device 110 or an inner wall of the bore 112 of the medical device 110. The image acquisition device 160 may be and/or include any suitable device that is capable of capturing image data of subjects located in a field of view of the image acquisition device. For example, the image acquisition device 160 may include a camera (e.g., a digital camera, an analog camera, etc.), a red-green-blue (RGB) sensor, an RGB-depth (RGB-D) sensor, or the like, or any combination thereof.

In some embodiments, the image acquisition device 160 may be mounted on any suitable positions. For example, the acquisition device 160 may be integrated into or mounted on the medical device 110 (e.g., the gantry 111, an inner wall of the bore of the medical device 110). In some embodiments, the image data acquired by the image acquisition device 160 may be transmitted to the processing device 120 for further analysis. Additionally or alternatively, the image data acquired by the image acquisition device 160 may be transmitted to a terminal device (e.g., the terminal(s) 140) for display and/or a storage device (e.g., the storage device 130) for storage.

It should be noted that the above description of the medical system 100 is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. For example, the medical system 100 may include one or more additional components. Additionally or alternatively, one or more components of the medical system 100, such as the image acquisition device 160 or the medical device 110 described above may be omitted. As another example, two or more components of the medical system 100 may be integrated into a single component. Merely by way of example, the processing device 120 (or a portion thereof) may be integrated into the medical device 110 or the image acquisition device 160.

FIG. 2 is a schematic diagram illustrating exemplary hardware and/or software components of a computing device 200 according to some embodiments of the present disclosure. The computing device 200 may be used to implement any component of the medical system 100 as described herein. For example, the processing device 120 and/or the terminal(s) 130 may be implemented on the computing device 200, respectively, via its hardware, software program, firmware, or a combination thereof. Although only one such computing device is shown, for convenience, the computer functions relating to the medical system 100 as described herein may be implemented in a distributed fashion on a number of similar platforms, to distribute the processing load. As illustrated in FIG. 2, the computing device 200 may include a processor 210, a storage device 220, an input/output (I/O) 230, and a communication port 240.

The processor 210 may execute computer instructions (e.g., program code) and perform functions of the processing device 120 in accordance with techniques described herein. The computer instructions may include, for example, routines, programs, objects, components, data structures, procedures, modules, and functions, which perform particular functions described herein.

Merely for illustration, only one processor is described in the computing device 200. However, it should be noted that the computing device 200 in the present disclosure may also include multiple processors, thus operations and/or method operations that are performed by one processor as described in the present disclosure may also be jointly or separately performed by the multiple processors. For example, if in the present disclosure the processor of the computing device 200 executes both operation A and operation B, it should be understood that operation A and operation B may also be performed by two or more different processors jointly or separately in the computing device 200 (e.g., a first processor executes operation A and a second processor executes operation B, or the first and second processors jointly execute operations A and B).

The storage device 220 may store data/information obtained from the medical device 110, the terminal(s) 130, the storage device 150, and/or any other component of the medical system 100. In some embodiments, the storage device 220 may store one or more programs and/or instructions to perform exemplary methods described in the present disclosure.

The I/O 230 may input and/or output signals, data, information, etc. In some embodiments, the I/O 230 may enable a user interaction with the processing device 120. In some embodiments, the I/O 230 may include an input device and an output device. The input information received through the input device may be transmitted to another component (e.g., the processing device 120) via, for example, a bus, for further processing.

The communication port 240 may be connected to a network (e.g., the network 120) to facilitate data communications. The communication port 240 may establish connections between the processing device 120 and the medical device 110, the terminal(s) 130, and/or the storage device 150. The connection may be a wired connection, a wireless connection, any other communication connection that can enable data transmission and/or reception, and/or any combination of these connections.

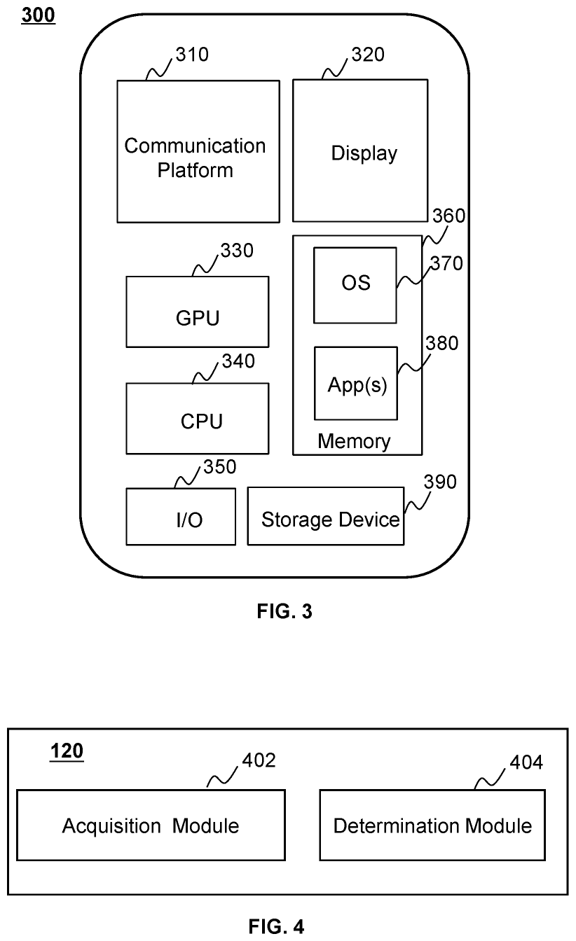

FIG. 3 is a schematic diagram illustrating exemplary hardware and/or software components of a mobile device 300 according to some embodiments of the present disclosure. In some embodiments, one or more components (e.g., a terminal 130 and/or the processing device 120) of the medical system 100 may be implemented on the mobile device 300.

As illustrated in FIG. 3, the mobile device 300 may include a communication platform 310, a display 320, a graphics processing unit (GPU) 330, a central processing unit (CPU) 340, an I/O 350, a memory 360, and a storage device 390. In some embodiments, any other suitable component, including but not limited to a system bus or a controller (not shown), may also be included in the mobile device 300. In some embodiments, a mobile operating system 370 (e.g., iOS™, Android™, Windows Phone™) and one or more applications 380 may be loaded into the memory 360 from the storage device 390 in order to be executed by the CPU 340. The applications 380 may include a browser or any other suitable mobile apps for receiving and rendering information relating to image processing or other information from the processing device 120. User interactions with the information stream may be achieved via the I/O 350 and provided to the processing device 120 and/or other components of the medical system 100 via the network 120.

To implement various modules, units, and their functionalities described in the present disclosure, computer hardware platforms may be used as the hardware platform(s) for one or more of the elements described herein. A computer with user interface elements may be used to implement a personal computer (PC) or any other type of work station or terminal device. A computer may also act as a server if appropriately programmed.

FIG. 4 is a block diagram illustrating an exemplary processing device 120 according to some embodiments of the present disclosure. In some embodiments, the processing device 120 may be implemented on a processing unit (e.g., a processor 210 illustrated in FIG. 2 or a CPU 340 as illustrated in FIG. 3). As shown in FIG. 4, the processing device 120 may include an acquisition module 402 and a determination module 404.

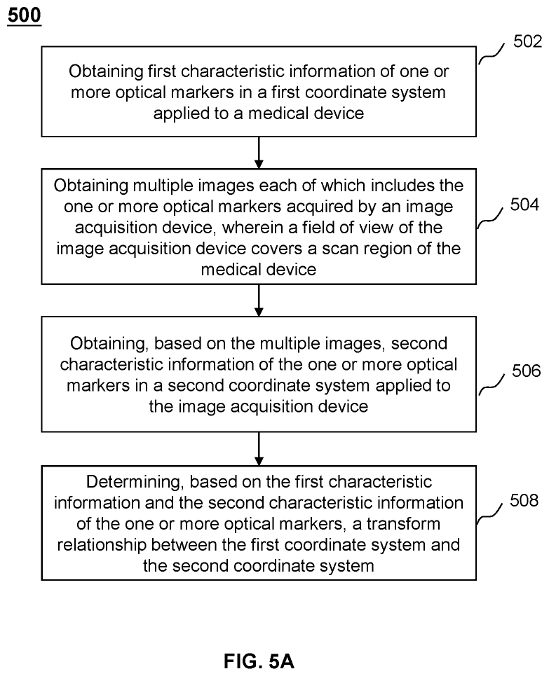

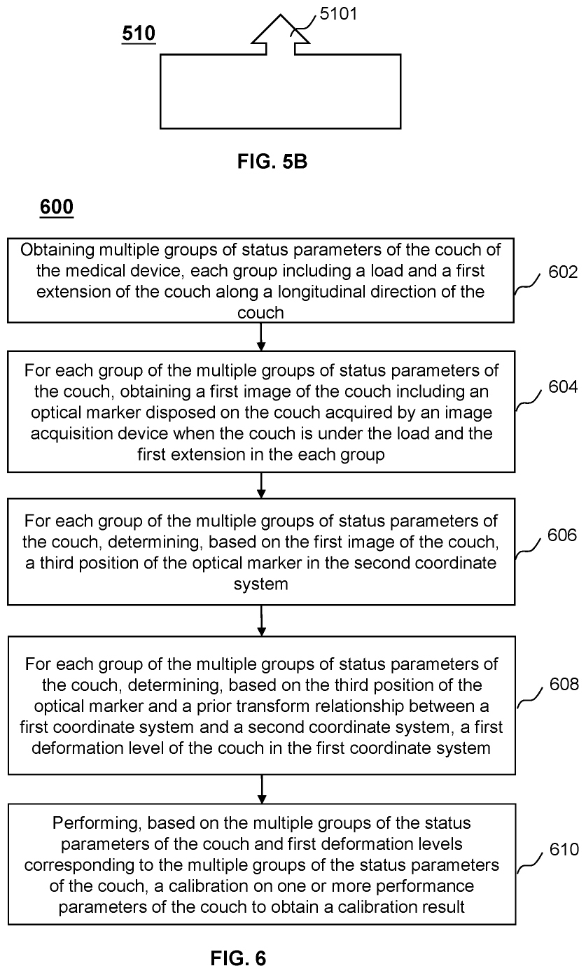

The acquisition module 402 may be configured to obtain information relating to the medical system 100. For example, the acquisition module 402 may obtain first characteristic information of one or more optical markers in a first coordinate system applied to a medical device. In some embodiments, the first characteristic information of the one or more optical markers may include at least one of first position information, first direction information, or first size information of each of at last a portion of the one or more optical markers in the first coordinate system. As another example, the acquisition module 402 may obtain multiple images each of which includes the one or more optical markers acquired by an image acquisition device, wherein a field of view of the image acquisition device covers a scan region of the medical device. As still another example, the acquisition module 402 may obtain second characteristic information of the one or more optical markers in a second coordinate system applied to images acquired by the image acquisition device based on the multiple images. In some embodiments, the second characteristic information of the one or more optical markers may include at least one of second position information, second direction information, or second size information of each of at last a portion of the one or more optical markers in the second coordinate system. More descriptions regarding the obtaining of the first characteristic information, the multiple images each of which includes the one or more optical markers, and the second characteristic information may be found elsewhere in the present disclosure. See, e.g., operations 502-506 in FIG. 5A, and relevant descriptions thereof.

The determination module 404 may be configured to determine a transform relationship between the first coordinate system and the second coordinate system based on the first characteristic information and the second characteristic information of the one or more optical markers. The transformation relationship may be used to perform a conversion between characteristic information denoted by the first coordinate system and characteristic information denoted by the second coordinate system. More descriptions regarding the determining of the transform relationship may be found elsewhere in the present disclosure. See, e.g., operation 508 in FIG. 5A, and relevant descriptions thereof.

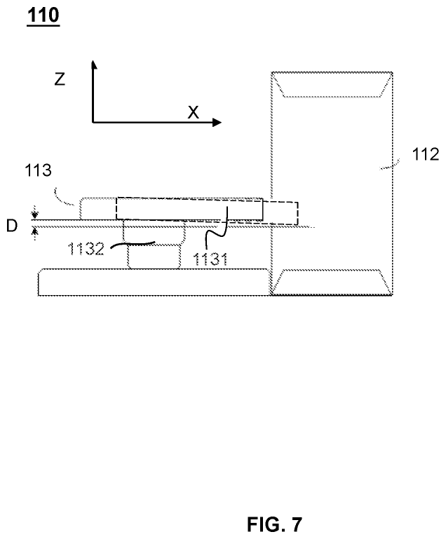

In some embodiments, the determination module 404 may be configured to calibrate one or more performance parameters of a couch of a medical device. More descriptions regarding the calibrating of one or more performance parameters of a couch of a medical device may be found elsewhere in the present disclosure. See, FIG. 6, and relevant descriptions thereof.

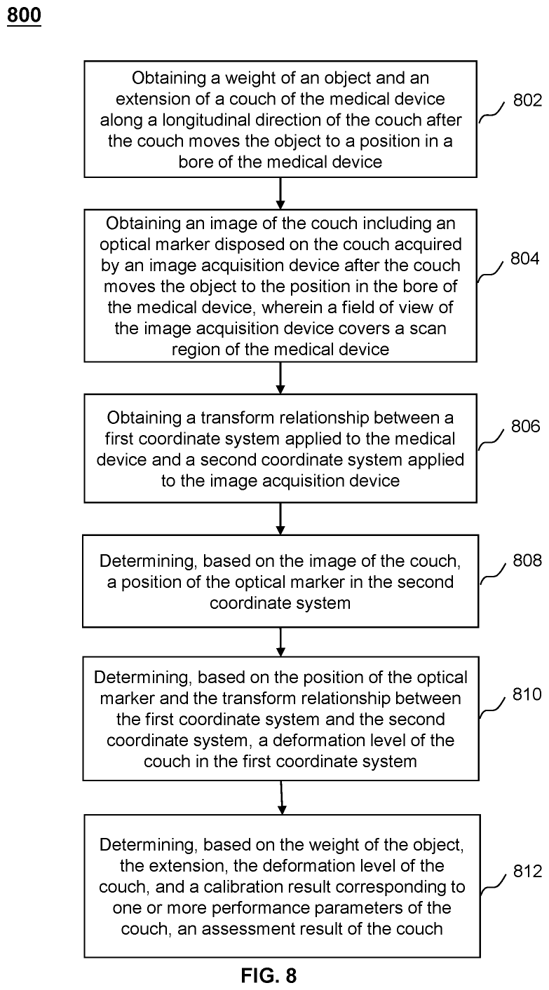

In some embodiments, the determination module 404 may be configured to determine an assessment result of a couch of a medical device. More descriptions regarding the determining of an assessment result of a couch of a medical device may be found elsewhere in the present disclosure. See, FIG. 8, and relevant descriptions thereof.

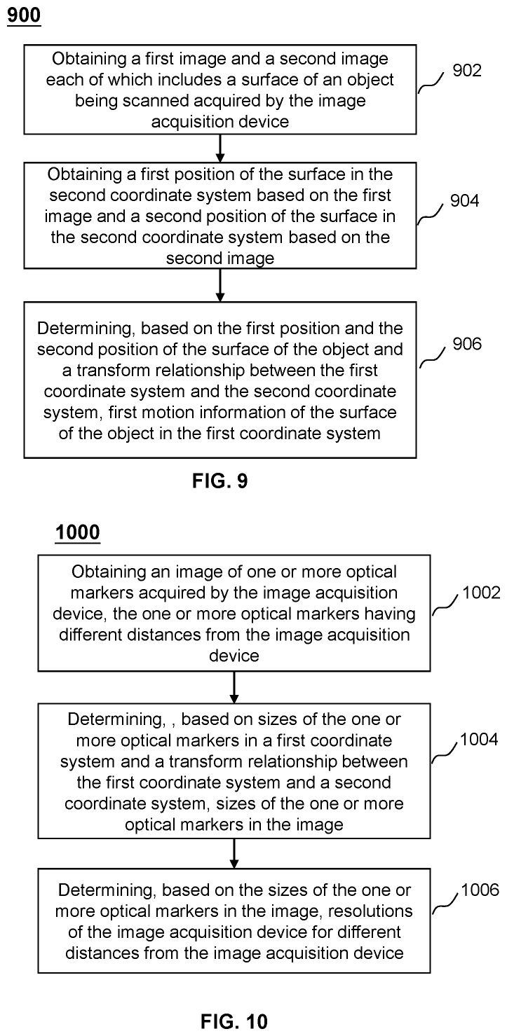

In some embodiments, the determination module 404 may be configured to determine motion information of a surface of an object being scanned in a first coordinate system applied to a medical device. More descriptions regarding the determining of motion information of a surface of an object being scanned in a first coordinate system applied to a medical device may be found elsewhere in the present disclosure. See, FIG. 9, and relevant descriptions thereof.

In some embodiments, the determination module 404 may be configured to determine resolutions of an image acquisition device of a medical system. More descriptions regarding the determining of resolutions of an image acquisition device of a medical system may be found elsewhere in the present disclosure. See, FIG. 10, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the processing device 120 may further include a storage module (not shown in FIG. 4). The storage module may be configured to store data generated during any process performed by any component of the processing device 120. As another example, each of at least some components of the processing device 120 may include a storage apparatus. Additionally or alternatively, at least some components of the processing device 120 may share a common storage apparatus.

FIG. 5A is a flowchart illustrating an exemplary process 500 for calibrating an image acquisition device of a medical system according to some embodiments of the present disclosure. In some embodiments, the process 500 may be implemented in the medical system 100 illustrated in FIG. 1. For example, the process 500 may be stored in a storage (e.g., the storage device 130, the storage device 220, the storage device 390) as a form of instructions, and invoked and/or executed by the processing device 120 (e.g., the processor 210 of the computing device 200 as illustrated in FIG. 2, the CPU 340 of the mobile device 300 as illustrated in FIG. 3, and/or one or more modules as illustrated in FIG. 4). The operations of the illustrated process presented below are intended to be illustrative. In some embodiments, the process 500 may be accomplished with one or more additional operations not described, and/or without one or more of the operations discussed. Additionally, the order in which the operations of the process 500 as illustrated in FIG. 5A and described below is not intended to be limiting.

In 502, the processing device 120 (e.g., the acquisition module 402) may obtain first characteristic information of one or more optical markers in a first coordinate system applied to a medical device.

In some embodiments, the first characteristic information of the one or more optical markers may include at least one of first position information, first direction information, or first size information of each of at last a portion of the one or more optical markers in the first coordinate system.

In some embodiments, the medical device may be a device (e.g., the medical device 110) configured to scan and/or treat a subject. The subject may include a biological subject and/or a non-biological subject. For example, the subject may include a specific portion of a body, such as the head, the thorax, the abdomen, or the like, or a combination thereof. As another example, the subject may be a man-made composition of organic and/or inorganic matters that are with or without life.

In some embodiments, the first coordinate system may be any multidimensional coordinate system (e.g., a word coordinate system, a three-dimension (3D) coordinate system including an X-axial, a Y-axial, and a Z axial as shown in FIG. 1) applied to the medical device. The first coordinate system may be set by a user via the terminal(s) 140 or according to a default setting of the medical system 100.

An optical marker refers to a marker that can reflect light. In some embodiments, an optical marker may include a 2D optical marker or a 3D optical marker. In some embodiments, an optical marker may include a specification defined by one or more parameters (e.g., characteristics) including a shape, a size, a color, a material, or the like, or a combination thereof. The shape may include a sphere, an ellipsoid, a cube, a wire, or other shapes. For example, an optical mark may be a marker including parallel or orthogonal short lines. The material may include a metal material, a resin material, a ceramic material, etc. In some embodiments, the density of the material may be greater than water. In some embodiments, the one or more optical markers may be in different specifications such that each of the one or more optical markers may be distinguished from each other. For example, a first optical marker and a second marker among the one or more optical markers may have shapes of a sphere and a cube respectively, such that the first optical marker may be distinguished with the second optical marker.

In some embodiments, the optical marker(s) may be disposed on suitable positions of the medical device before or after the medical device is installed in an examination room. For example, the optical marker(s) may be disposed on the medical device as factory settings in advance. In some embodiments, the optical marker(s) may be disposed on at least one of a couch of the medical device (e.g., the couch 113 of the medical device 110 described in FIG. 1) or an inner wall of a bore of the medical device (e.g., the bore 112 of the medical device 110 described in FIG. 1).

In some embodiments, at least one of the optical marker(s) may be disposed on the couch of the medical device (e.g., an upper surface of a table top of the couch), and the remaining of the optical marker(s) may be disposed on the inner wall of the bore of the medical device. In some embodiments, if all the optical marker(s) are disposed on the inner wall of the bore of the medical device, the optical marker(s) may be multiple optical markers. The multiple optical markers may be disposed different positions of the inner wall of the bore of the medical device. For example, the multiple optical markers may be arranged at the inner wall of the bore of the medical device in multiple rows along a circumferential direction of the bore. Each of the multiple rows of the multiple optical markers may include at least one optical marker.

The first position information of the one or more optical markers refers to information relating to positions of the one or more optical markers in the first coordinate system. For example, the first position information of the one or more optical markers may include multiple first positions of the one or more optical markers in the first coordinate system. In some embodiments, a first position of an optical marker in the first coordinate system may be denoted by a position of a physical point of the optical marker in the first coordinate system. For example, a first position of an optical marker in the first coordinate system may be denoted by a coordinate of a physical point located at the center of the optical marker.

The first direction information of an optical marker refers to information relating to directions of the optical marker in the first coordinate system. In some embodiments, a portion of an optical marker may be configured to indicate the direction of the optical marker. The first direction information of the optical marker may include an orientation of the portion of the optical marker in the first coordinate system. In some embodiments, the orientation of the portion of the optical marker in the first coordinate system may be denoted by an angle between the optical marker and each coordinate axis in the first coordinate system. For example, FIG. 5B is a schematic diagram illustrating an exemplary optical marker 510 according to some embodiments of the present disclosure. As shown in FIG. 5B, a region 5101 with an arrow shape of the optical marker 510 may be configured to indicate the direction of the optical marker 510. The first direction information of the optical marker 510 may include a direction of the region 5101 of the optical marker in the first coordinate system.

The first size information of the one or more optical markers refers to information relating to sizes of the one or more optical markers in the first coordinate system. For example, the first size information of the one or more optical markers may include physical sizes of the one or more optical markers in the first coordinate system.

In some embodiments, the first characteristic information of the multiple optical markers in the first coordinate system may be obtained from, for example, a prior measurement. For example, the first characteristic information of the multiple optical markers in the first coordinate system may be previously determined and stored in a storage device (e.g., the storage device 130, the storage device 220, the storage device 390, or an external source). The processing device 120 may retrieve the first characteristic information of the multiple optical markers from the storage.

In some embodiments, if an optical marker among the one or more optical markers is disposed on the couch of the medical device, the processing device 120 may obtain positions of the couch in the first coordinate system during movement of the couch. The processing device 120 may determine the first position information of the optical marker in the first coordinate system based on the positions of the couch in the first coordinate system. Merely by way of example, an initial first position of the optical marker in the first coordinate system when the couch is located at an initial position of the couch may be obtained from, for example, a prior measurement. The processing device 120 may sequentially obtain multiple positions of the couch in the first coordinate system during movement of the couch. For each of the multiple positions of the couch in the first coordinate system, the processing device 120 may obtain movement data of the couch when the couch moves from a prior position of the position of the couch to the position of the couch. The movement data of the couch may include, for example, a displacement of the couch, a speed of the couch, an acceleration of the couch, a movement direction of the couch, etc. As used herein, the displacement of the couch may refer to a location change in space when the couch moves from one position to another position. The processing device 120 may determine multiple first positions of the optical marker corresponding to the multiple positions of the couch based on the multiple displacements of the couch and the initial first position of the optical marker. In some embodiments, the displacements of the couch may be acquired directly from a device for controlling the couch. In some embodiments, the displacement of the couch may be determined based on other movement data (e.g., the speed of the couch, the acceleration of the couch, the movement direction of the couch, etc.) by the processing device 120. For example, the acceleration of the couch may be acquired by a measurement device (e.g., an acceleration sensor). The processing device 120 may process the acceleration of the couch by performing a double integral on the acceleration of the couch to determine the displacement of the couch.

In 504, the processing device 120 (e.g., the acquisition module 402) may obtain multiple images each of which includes the one or more optical markers acquired by an image acquisition device, wherein a field of view of the image acquisition device covers a scan region of the medical device.

The image acquisition device may be configured to capture image data of a region in the bore of the medical device (e.g., the scan region of the medical device). The image acquisition device may be any suitable device (e.g., the image acquisition device 160) that is capable of capturing image data subjects located in the field of view of the image acquisition device. The one or more optical markers may be located in the field of view of the image acquisition device, so that the image acquisition device may acquire the multiple images each of which includes the one or more optical markers.

In some embodiments, after the multiple images are acquired by the image acquisition device, the multiple images may be directly transmitted to the processing device 120. Alternatively, the multiple images may be acquired and stored in a storage device (e.g., the storage device 130, the storage device 220, the storage device 390, or an external source). The processing device 120 may retrieve the multiple images from the storage.

In 506, the processing device 120 (e.g., the acquisition module 402) may obtain, based on the multiple images, second characteristic information of the one or more optical markers in a second coordinate system applied to images acquired by the image acquisition device.

In some embodiments, the second coordinate system may refer to an image coordinate system (e.g., a pixel coordinate system, a physical image coordinate system). In some embodiments, the second coordinate system may be constructed based on a coordinate system (also referred to as a third coordinate system) applied to the image acquisition device. For example, the second coordinate system may be a coordinate system formed by projecting the coordinate system applied to the image acquisition device onto a certain plane along the optical axis of the image acquisition device. The coordinate system applied to the image acquisition device may be set by a user via the terminal(s) 140 or according to a default setting of the medical system 100. For example, the second coordinate system may be a 3D rectangular coordinate system with an optical center of the image acquisition device as the origin and the optical axis as one coordinate axis.

In some embodiments, the second characteristic information of the one or more optical markers may include at least one of second position information, second direction information, or second size information of each of at last a portion of the one or more optical markers in the second coordinate system.

In some embodiments, the second position information of the one or more optical markers refers to information relating to positions of the one or more optical markers in the second coordinate system. For example, the second position information of the one or more optical markers may include multiple second positions of the one or more optical markers in the second coordinate system. In some embodiments, an optical marker may be represented in an image as a target region. A second position of an optical marker in the second coordinate system may be denoted by a position of a target region corresponding to the optical marker in the image. A position of a target region in an image may be denoted by a position of a reference point (e.g., a pixel or a voxel) of the target region in the second coordinate system. For example, the position of the target region in the image may be denoted by a coordinate of a pixel or a voxel located at the center of the target region in the second coordinate system. In some embodiments, if the one or more optical markers are a plurality of optical markers, the determination of the second positions of the plurality of optical markers may include determining and/or identifying the plurality of optical markers from each image based on the target regions in the image corresponding to the plurality of optical markers. For example, the plurality of optical markers may have different specifications (e.g., shapes, materials, etc.). The plurality of optical markers may be identified based on the target regions corresponding to the plurality of optical markers in the image and the specifications of the plurality of optical markers. Then, the second positions of the plurality of optical markers may be determined.

The second direction information of the one or more optical markers refers to information relating to directions of the one or more optical markers in the second coordinate system. In some embodiments, a portion of an optical marker may be configured to indicate the direction of the optical marker. The second direction information of the optical marker may include an orientation of the portion of the optical marker in the second coordinate system. In some embodiments, the orientation of the portion of the optical marker in the second coordinate system may be denoted by an angle between the portion of the optical marker and each coordinate axis in the second coordinate system.

In some embodiments, an optical marker may be represented in an image as a target region. The second size information of an optical marker in an image refers to information relating to a size of a target region corresponding to the optical marker in the image (e.g., a physical size, a pixel size, etc.).

In some embodiments, the processing device 120 may determine the second characteristic information of the multiple optical markers in the second coordinate system by analyzing the multiple images. For example, the processing device 120 may determine the second characteristic information of the multiple optical markers in the second coordinate system using a feature extraction algorithm. Exemplary feature extraction algorithms may include a scale invariant feature transform (SIFT) algorithm, an average amplitude difference function (AMDF) algorithm, a histogram of gradient (HOG) algorithm, a speeded up robust features (SURF) algorithm, a local binary pattern (LBP) algorithm, etc. Alternatively, the processing device 120 may transmit the multiple images to another computing device. The computing device may determine the second characteristic information of the multiple optical markers in the second coordinate system and transmit the second characteristic information back to the processing device 120. In some embodiments, the second characteristic information of the multiple optical markers in the second coordinate system may be previously determined and stored in a storage device (e.g., the storage device 130, the storage device 220, the storage device 390, or an external source). The processing device 120 may retrieve the second characteristic information of the multiple optical markers from the storage.

In 508, the processing device 120 (e.g., the determination module 404) may determine, based on the first characteristic information and the second characteristic information of the one or more optical markers, a transform relationship between the first coordinate system and the second coordinate system.

The transformation relationship may be used to perform a conversion between characteristic information denoted by the first coordinate system and characteristic information denoted by the second coordinate system. For example, the transformation relationship may be used to perform a conversion between position information denoted by the first coordinate system and position information denoted by the second coordinate system. As another example, the transformation relationship may be used to perform a conversion between direction information denoted by the first coordinate system and direction information denoted by the second coordinate system. As still another example, the transformation relationship may be used to perform a conversion between size information denoted by the first coordinate system and size information denoted by the second coordinate system. In some embodiments, the transformation relationship may be represented by, for example, a transformation equation or algorithm. The transformation relationship (e.g., a transformation equation or algorithm) may be defined by one or more parameters (also referred to as mapping parameters) relating to the image acquisition device. In some embodiments, the parameters relating to the image acquisition device may include extrinsic parameters and/or intrinsic parameters. Exemplary extrinsic parameters may include translation parameters, rotation parameters, etc., which may be used to determine a position and a direction of the image acquisition device in a 3D space. Exemplary intrinsic parameters may include a focal length, a pixel size, a distortion parameter, of the image acquisition device. In the present disclosure, the calibration of the image acquisition device may refer to that determining, adjusting, or updating values of one or more parameters (e.g., extrinsic parameters, intrinsic parameters) relating to the image acquisition device.

In some embodiments, the processing device 120 may determine the transform relationship between the first coordinate system and the second coordinate system based on the first characteristic information and the second characteristic information of the one or more optical markers to achieve the calibration of the parameters relating to the image acquisition device. Merely by way of example, the processing device 120 may determine a function including the parameters relating to the image acquisition device whose values are unknown. The processing device 120 may determine multiple characteristic pairs based on the first characteristic information and the second characteristic information of the one or more optical markers. Each pair of the multiple characteristic pairs may include first characteristic information and second characteristic information of an optical marker obtained under a condition that the optical marker remains the same, that is, a size, a position, and a direction, etc., of the optical marker remain unchanged. For example, a characteristic pair may include a first position in the first coordinate system and a second position in the second coordinate system of an optical marker. As another example, a characteristic pair may include a first direction in the first coordinate system and a second direction in the second coordinate system of an optical marker. As still another example, a characteristic pair may include a first size in the first coordinate system and a second size in the second coordinate system of an optical marker. The processing device 120 may determine the values of the parameters relating to the image acquisition device in function based on multiple characteristic pairs to achieve the calibration of the parameters relating to the image acquisition device. Further, the processing device 120 may determine the transform relationship between the first coordinate system and the second coordinate system according to the obtained values of the parameters relating to the image acquisition device.

In some embodiments, the processing device 120 may determine installation position information of the image acquisition device in the first coordinate system based on the calibrated parameters relating to the image acquisition device. For example, the processing device 120 may determine coordinates corresponding to the installation position in the first coordinate system according to the translation parameters relating to the image acquisition device. The processing device 120 may determine orientation information of the image acquisition device in the first coordinate system according to the rotation parameters relating to the image acquisition device. When the image acquisition device needs to be reinstalled, the image acquisition device may be reinstalled based on the coordinates corresponding to the installation position and the orientation information of the image acquisition device. When the image acquisition device needs to be replaced, a new image acquisition device may be installed based on the coordinates corresponding to the installation position, the orientation information of the image acquisition device, and one or more parameters (e.g., a shape, a size, a field of view, etc., of the image acquisition device) of the new image acquisition device. In some embodiments, after the image acquisition device is replaced, a calibration of the new image acquisition device may be simplified. For example, the calibration of the new image acquisition device may be performed using a method with a relatively low accuracy. In this way, the efficiency of the calibration of the new image acquisition device may be improved.

According to some embodiments of the present disclosure, the calibration of the parameters relating to the image acquisition device may be achieved automatically based on the one or more optical markers disposed on the medical device, which does not need to manually adjust the one or more optical markers to obtain the multiple images of the one or more optical markers at multiple different positions, thereby shorting time-consuming, reducing manual intervention, and improving the efficiency of the calibration of the parameters relating to the image acquisition device.

In some embodiments, the calibration of the parameters relating to the image acquisition device may be performed periodically or aperiodically as needed. For example, the calibration of the parameters relating to the image acquisition device may be automatically performed every time medical device is turned on. Therefore, after the image acquisition device is replaced or moved, there is no need for a user to manually perform additional calibration on the parameters relating to the image acquisition device.

FIG. 6 is a flowchart illustrating an exemplary process 600 for calibrating one or more performance parameters of a couch of a medical device according to some embodiments of the present disclosure. In some embodiments, the process 600 may be implemented in the medical system 100 illustrated in FIG. 1. For example, the process 600 may be stored in a storage (e.g., the storage device 150, the storage device 220, the storage device 390) as a form of instructions, and invoked and/or executed by the processing device 120 (e.g., the processor 210 of the computing device 200 as illustrated in FIG. 2, the CPU 340 of the mobile device 300 as illustrated in FIG. 3, and/or one or more modules as illustrated in FIG. 4). The operations of the illustrated process presented below are intended to be illustrative. In some embodiments, the process 600 may be accomplished with one or more additional operations not described, and/or without one or more of the operations discussed. Additionally, the order in which the operations of the process 600 as illustrated in FIG. 6 and described below is not intended to be limiting.

In 602, the processing device 120 (e.g., the determination module 404) may obtain multiple groups of status parameters of the couch of the medical device, each group including a load and a first extension of the couch along a longitudinal direction of the couch.

In some embodiments, the medical device may be a device (e.g., the medical device 110) configured to scan and/or treat a subject. The couch (e.g., the couch 113) may be configured to support and/or transfer a subject to, for example, a scanning region or a treatment region of the medical device. More descriptions for the medical device and the couch may be found elsewhere in the present disclosure (e.g., FIG. 1 and the descriptions thereof).

In some embodiments, the load of the couch refers to a weight of a subject supported by a table top of the couch (e.g., the table top 1131 of the couch 113). The table top of the couch may extend to a plurality of positions along the longitudinal direction (e.g., the X direction as shown in FIG. 1) of the couch. An extension of the couch along the longitudinal direction of the couch refers to a length that the table top of the couch extends along the longitudinal direction of the couch.

The multiple groups of status parameters of the couch may be set manually by a user (e.g., an engineer) or determined by the processing device 120. In some embodiments, some groups of the multiple groups of status parameters may include a same load and different first extensions. In some embodiments, some groups of the multiple groups of status parameters may include different loads and a same first extension. In some embodiments, some groups of the multiple groups of status parameters may include different loads and different first extensions.

In 604, for each group of the multiple groups of status parameters of the couch, the processing device 120 (e.g., the determination module 404) may obtain a first image of the couch including an optical marker disposed on the couch acquired by an image acquisition device when the couch is under the load and the first extension in the group.

The image acquisition device may be configured to capture image data of a region in a bore of the medical device (e.g., a scan region of the medical device). The image acquisition device may be any suitable device (e.g., the image acquisition device 160) that is capable of capturing image data subjects located in a field of view of the image acquisition device. The optical marker refers to a marker that can reflect light. The optical marker may be disposed on a suitable position of the couch (e.g., an end of couch near the bore of the medical device). More descriptions for the image acquisition device and the optical marker may be found elsewhere in the present disclosure (e.g., FIG. 1, FIG. 5A, and the descriptions thereof). In some embodiments, the obtaining of the first image of the couch may be performed in a similar manner as that of the multiple images each of which includes the one or more optical markers as described in connection with operation 504, and the descriptions of which are not repeated here.

In 606, for each group of the multiple groups of status parameters of the couch, the processing device 120 (e.g., the determination module 404) may determine, based on the first image of the couch, a third position of the optical marker in the second coordinate system.

In some embodiments, the determining of the third position of the optical marker in the second coordinate system may be performed in a similar manner as that of second characteristic information of an optical marker in the second coordinate system as described in connection with operation 506, and the descriptions of which are not repeated here.

In 608, for each group of the multiple groups of status parameters of the couch, the processing device 120 (e.g., the determination module 404) may determine, based on the third position of the optical marker and a prior transform relationship between a first coordinate system and a second coordinate system, a first deformation level of the couch in the first coordinate system.

The table top of the couch may extend or retract to a plurality of positions along the longitudinal direction (e.g., the X direction as shown in FIG. 1) of the couch. When the table top of the couch moves to different positions along the longitudinal direction of the couch, the couch may deform (e.g., deflect or sag) to generate the first deformation level of the couch in the first coordinate system. For example, as shown in FIG. 7, FIG. 7 is a side view of the medical device 110 in FIG. 1 according to some embodiments of the present disclosure. As shown in FIG. 7, when the supporting component 1132 for supporting the table top 1131 of the couch 113 is located at an end of the couch 113 away from the bore 112 of the medical device 110, the table top 1131 may deform to generate the first deformation level of the couch 113 in the first coordinate system when the table top 1131 moves to different positions along the longitudinal direction (i.e., the X direction shown in the FIG. 7) of the couch 113.

In some embodiments, the first coordinate system and the second coordinate system may be the same as or similar to the first coordinate system and the second coordinate system described in FIG. 5A. In some embodiments, the calibration on the parameters relating to the image acquisition device may be performed periodically or aperiodically as needed, and the obtained multiple transform relationships between the first coordinate system and the second coordinate system may be stored in a storage device (e.g., the storage device 130, the storage device 220, the storage device 390, or an external source). The processing device 120 may retrieve a prior transform relationship between the first coordinate system and the second coordinate system from the storage. For example, the processing device 120 may retrieve a prior transform relationship that was determined at the latest from the storage.

In some embodiments, the processing device 120 may determine a fourth position of the optical marker in the first coordinate system based on the third position of the optical marker and the prior transform relationship between the first coordinate system and the second coordinate system. In some embodiments, the processing device 120 may determine the first deformation level of the couch in the first coordinate system based on the fourth position of the optical marker. In some embodiments, the first deformation level may include a sag level along a vertical direction. A sag level of the table top may be defined by an offset in a vertical direction when the table top is under no load and extension and when the couch is under the load and the first extension.

In some embodiments, the optical marker may be disposed on an end of the table top near the bore of the medical device. The processing device 120 may determine a first position of the end of the table top near the bore of the medical device in the first coordinate system when the couch is under the load and the first extension based on the fourth position of the optical marker. For example, the processing device 120 may designate the fourth position of the optical marker as the first position of the end of the table top near the bore of the medical device. The processing device 120 may obtain a second position of the end of the table top near the bore of the medical device in the first coordinate system when the couch is under no load and extension. The processing device 120 may determine the offset between the first position and the second portion of the end of the table top near the bore of the medical device as the sag level of the table top. For example, as shown in FIG. 7, the table top 1131 is located at the solid line area when the table top 1131 is under no load and extension, and the table top 1131 is located at the dotted line area when the couch is under the load and the first extension. The sag level of the table top 1131 may be the distance D in the vertical direction (i.e., the Z direction) between an end of the table top 1131 near the bore of the medical device when the table top is under no load and extension and the end of the table top 1131 near the bore of the medical device when the table top is under the load and the first extension.

In 610, the processing device 120 (e.g., the determination module 404) may perform, based on the multiple groups of the status parameters of the couch and first deformation levels corresponding to the multiple groups of the status parameters of the couch, a calibration on one or more performance parameters of the couch to obtain a calibration result.

In some embodiments, the one or more performance parameters of the couch may include one or more parameters relating to a deformation level of the couch, such as a sag level along a vertical direction. For each of the one or more performance parameters of the couch, the processing device 120 may determine a relationship between the performance parameter and the status parameters of the couch based on the multiple groups of the status parameters of the couch and first deformation levels corresponding to the multiple groups of the status parameters of the couch to perform the calibration on the performance parameter of the couch. For example, the processing device 120 may determine a relationship between the sag level and the status parameters of the couch based on the multiple groups of the status parameters of the couch and the sag levels corresponding to the multiple groups of the status parameters of the couch to perform the calibration on the sag level of the couch. Merely by way of example, the processing device 120 may perform a fitting on the multiple groups of the status parameters of the couch and first deformation levels corresponding to the multiple groups of the status parameters of the couch to obtain a function or a model, and the obtained function or the model may be determined as the relationship between the performance parameter and the status parameters of the couch. The processing device 120 may designate the relationship between the performance parameter and the status parameters of the couch as the calibration result corresponding to the performance parameter.

According to some embodiments of the present disclosure, the calibration of the one or more performance parameters of the couch may be achieved automatically based on the optical marker disposed on the couch and the transform relationship between the first coordinate system and the second coordinate system, which does not need to manually obtain the first deformation levels corresponding to the multiple groups of the status parameters of the couch, thereby shorting time-consuming, reducing manual intervention, and improving the efficiency of the calibration of the one or more performance parameters of the couch.

FIG. 8 is a flowchart illustrating an exemplary process 800 for determining an assessment result of a couch of a medical device according to some embodiments of the present disclosure. In some embodiments, the process 800 may be implemented in the medical system 100 illustrated in FIG. 1. For example, the process 800 may be stored in a storage (e.g., the storage device 150, the storage device 220, the storage device 390) as a form of instructions, and invoked and/or executed by the processing device 120 (e.g., the processor 210 of the computing device 200 as illustrated in FIG. 2, the CPU 340 of the mobile device 300 as illustrated in FIG. 3, and/or one or more modules as illustrated in FIG. 4). The operations of the illustrated process presented below are intended to be illustrative. In some embodiments, the process 800 may be accomplished with one or more additional operations not described, and/or without one or more of the operations discussed. Additionally, the order in which the operations of the process 800 as illustrated in FIG. 8 and described below is not intended to be limiting.

In 802, the processing device 120 (e.g., the determination module 404) may obtain a weight of an object and an extension (also referred to as a second extension) of a couch of the medical device along a longitudinal direction of the couch after the couch moves the object to a position in a bore of the medical device.

In some embodiments, the medical device may be a device (e.g., the medical device 110) configured to scan and/or treat a subject. The couch (e.g., the couch 113) may be configured to support and/or transfer a subject to, for example, a scanning region or a treatment region of the medical device. More descriptions for the medical device and the couch may be found elsewhere in the present disclosure (e.g., FIG. 1 and the descriptions thereof). In some embodiments, the object may include a biological object and/or a non-biological object.

In some embodiments, the processing device 120 may obtain the weight of the object before the object is located at the couch using a measurement apparatus (e.g., a weighing scale). In some embodiments, the after the couch moves the object to the position in the bore of the medical device, the processing device 120 may obtain the extension of the couch according to a length that a table top of the couch extends along the longitudinal direction of the couch. More descriptions for the extension of the couch may be found elsewhere in the present disclosure (e.g., FIG. 6 and the descriptions thereof).

In 804, the processing device 120 (e.g., the determination module 404) may obtain an image of the couch (e.g., also referred to as a second image of the couch) including an optical marker disposed on the couch acquired by an image acquisition device after the couch moves the object to the position in the bore of the medical device, wherein a field of view of the image acquisition device covers a scan region of the medical device.

The image acquisition device may be configured to capture image data of a region in the bore of the medical device (e.g., a scan region of the medical device). The image acquisition device may be any suitable device (e.g., the image acquisition device 160) that is capable of capturing image data subjects located in a field of view of the image acquisition device. The optical marker refers to a marker that can reflect light. The optical marker may be disposed on a suitable position of the couch (e.g., an end of couch near the bore of the medical device). More descriptions for the image acquisition device and the optical marker may be found elsewhere in the present disclosure (e.g., FIG. 1, FIG. 5A, and the descriptions thereof). In some embodiments, the obtaining of the image of the couch may be performed in a similar manner as that of the multiple images each of which includes the one or more optical markers as described in connection with operation 504, and the descriptions of which are not repeated here.

In 806, the processing device 120 (e.g., the determination module 404) may obtain a transform relationship between a first coordinate system applied to the medical device and a second coordinate system applied to the image acquisition device.

In some embodiments, the first coordinate system and the second coordinate system may be the same as or similar to the first coordinate system and the second coordinate system described in FIG. 5A. In some embodiments, the processing device 120 may obtain the transform relationship between the first coordinate system and the second coordinate system according to some embodiments described in the process 500. In some embodiments, the processing device 120 may obtain the transform relationship between the first coordinate system and the second coordinate system from a storage device (e.g., the storage device 130, the storage device 220, the storage device 390, or an external source). In some embodiments, the calibration on the parameters relating to the image acquisition device may be performed periodically or aperiodically as needed, and the obtained multiple transform relationships between the first coordinate system and the second coordinate system may be stored in the storage device. The processing device 120 may retrieve a prior transform relationship between the first coordinate system and the second coordinate system from the storage. For example, the processing device 120 may retrieve a prior transform relationship that was determined at the latest from the storage.

In 808, the processing device 120 (e.g., the determination module 404) may determine, based on the image, a position of the optical marker in the second coordinate system.

In some embodiments, the operation 808 may be similar to or the same as the operation 606 of the process 600 as illustrated in FIG. 6.

In 810, the processing device 120 (e.g., the determination module 404) may determine, based on the position of the optical marker and the transform relationship between the first coordinate system and the second coordinate system, a deformation level of the couch (also referred to as a second deformation level of the couch) in the first coordinate system.

In some embodiments, the deformation level may include a sag level along a vertical direction. In some embodiments, the operation 810 may be similar to or the same as the operation 608 of the process 600 as illustrated in FIG. 6.

In 812, the processing device 120 (e.g., the determination module 404) may determine, based on the weight of the object, the extension, the deformation level of the couch, and a calibration result corresponding to one or more performance parameters of the couch, an assessment result of the couch.

In some embodiments, the processing device 120 may determine status parameters of the couch based on the weight of the object and the extension of the couch. In some embodiments, the status parameters of the couch may include a load and an extension of the couch along the longitudinal direction of the couch. For example, the processing device 120 may designate the weight of the object and the extension of the couch as a load and an extension of the couch in the status parameters. The processing device 120 may assess the performance or status of the couch based on the status parameters, the calibration result corresponding to the one or more performance parameters of the couch, and the deformation level of the couch.

In some embodiments, the one or more performance parameters of the couch may include one or more parameters relating to a deformation level of the couch, such as a sag level along a vertical direction. The calibration result corresponding to the one or more performance parameters of the couch may be configured to indicate a relationship between each of the one or more performance parameters and status parameters of the couch. In some embodiments, the processing device 120 may obtain the calibration result according to some embodiments described in the process 600. In some embodiments, the processing device 120 may obtain the calibration result from a storage device (e.g., the storage device 130, the storage device 220, the storage device 390, or an external source). In some embodiments, the processing device 120 may obtain a reference deformation level of the couch according to the status parameter and the calibration result.

In some embodiments, the assessment result of the couch may include an assessment result relating to a performance, an assessment result relating to the calibration result, an assessment result relating to a status, of the couch, or the like, or any combination thereof.

In some embodiments, the processing device 120 may determine the assessment result relating to the performance of the couch based on the deformation level of the couch. For example, the processing device 120 may determine whether the performance of the couch satisfies requirements for use based on the deformation level of the couch. For example, the processing device 120 may determine whether the deformation level of the couch is smaller than a first threshold. In response to determining that the deformation level of the couch is not smaller than the first threshold, the processing device 120 may determine that the performance of the couch does not satisfy requirements for use. In some embodiments, after it is determined that the performance of the couch does not satisfy requirements for use, the processing device 120 may send a prompt massage to a user terminal (e.g., the terminal 140) to prompt that the couch needs to be repaired or replaced. In response to determining that the deformation level of the couch is smaller than the first threshold, the processing device 120 may determine that the performance of the couch satisfies requirements for use.

In some embodiments, the processing device 120 may determine the assessment result relating to the performance of the couch and/or the assessment result relating to the calibration result of the couch based on the deformation level of the couch and the reference deformation level of the couch. For example, for each of the one or more performance parameters, the processing device 120 may determine a difference between a value of the performance parameter in the deformation level and a value of the performance parameter in the reference deformation level. The processing device 120 may determine whether the difference is greater than a second threshold. In response to determining that the difference is greater than the second threshold, the processing device 120 may determine the couch does not satisfy requirements for use. The processing device 120 may send a prompt massage to a user terminal (e.g., the terminal 140) to prompt that the couch needs to be repaired or replaced. In response to determining that the difference is not greater than the second threshold, the processing device 120 may determine whether the difference is greater than a third threshold smaller than the second threshold. In response to determining that the difference is greater than the third threshold, the processing device 120 may determine that the calibration result corresponding to the performance parameter is invalid. In this case, the processing device 120 may update the calibration result corresponding to the performance parameter with the value of the performance parameter in the deformation level. In response to determining that the difference is not greater than the third threshold, the processing device 120 may determine that the calibration result corresponding to the performance parameter is valid.

In some embodiments, the processing device 120 may determine the assessment result relating to the status of the couch based on the deformation level of the couch and the reference deformation level of the couch. For example, the processing device 120 may determine whether the status of the couch needs to be adjusted based on the deformation level of the couch and the reference deformation level of the couch. In some embodiments, the adjusting of the status of the couch may include an adjusting of a position of the couch, for example, an adjusting of the extension, an adjusting of a height of the couch, etc. Merely by way of example, the processing device 120 may determine a sag level difference between a value of the sag level in the deformation level and a value of the sag level in the reference deformation level. the processing device 120 may determine whether the sag level difference in the deformation level is greater than a fourth threshold. In response to determining that the sag level difference is greater than the fourth threshold, the processing device 120 may determine that the height of the couch needs to be adjusted. In some embodiments, after it is determined that the height of the couch needs to be adjusted, the processing device 120 may send a prompt massage to a user terminal (e.g., the terminal 140) to prompt that the height of the couch needs to be adjusted. In response to determining that the sag level difference is not greater than the fourth threshold, the processing device 120 may determine that the height of the couch does not need to be adjusted.

According to some embodiments of the present disclosure, the performance or status of the couch may be assessed automatically based on the optical marker disposed on the couch and the transform relationship between the first coordinate system and the second coordinate system, which is simple and has a high efficiency and accuracy, thereby ensuring the accuracy of the subsequent scan.

FIG. 9 is a flowchart illustrating an exemplary process 900 for determining motion information of a surface of an object being scanned in a first coordinate system applied to a medical device according to some embodiments of the present disclosure. In some embodiments, the process 900 may be implemented in the medical system 100 illustrated in FIG. 1. For example, the process 900 may be stored in a storage (e.g., the storage device 150, the storage device 220, the storage device 390) as a form of instructions, and invoked and/or executed by the processing device 120 (e.g., the processor 210 of the computing device 200 as illustrated in FIG. 2, the CPU 340 of the mobile device 300 as illustrated in FIG. 3, and/or one or more modules as illustrated in FIG. 4). The operations of the illustrated process presented below are intended to be illustrative. In some embodiments, the process 900 may be accomplished with one or more additional operations not described, and/or without one or more of the operations discussed. Additionally, the order in which the operations of the process 900 as illustrated in FIG. 9 and described below is not intended to be limiting.

In 902, the processing device 120 (e.g., the determination module 404) may obtain a first image and a second image each of which includes a surface of an object being scanned acquired by the image acquisition device.

Regarding descriptions for the image acquisition device and the medical device may be found elsewhere in the present disclosure (e.g., FIG. 1, FIG. 5A, and the descriptions thereof). In some embodiments, the obtaining of the first image and the second image each of which includes the surface of the object may be performed in a similar manner as that of the multiple images each of which includes the one or more optical markers as described in connection with operation 504, and the descriptions of which are not repeated here.

In 904, the processing device 120 (e.g., the determination module 404) may obtain a first position of the surface in a second coordinate system based on the first image and a second position of the surface in the second coordinate system based on the second image.

Regarding descriptions for the first coordinate system and the second coordinate system may be found elsewhere in the present disclosure (e.g., FIG. 5A and the descriptions thereof).

In some embodiments, a position of the surface in an image may be denoted by a position of a reference point (e.g., a pixel or a voxel) or a portion (e.g., a target region) of the surface in the second coordinate system. The processing device 120 may obtain a first position of the reference point (or the portion) of the surface in the second coordinate system based on the first image and designate the first position of the reference point (or the portion) of the surface as the first position of the surface in the second coordinate system. The processing device 120 may obtain a second position of the reference point (or the portion) of the surface in the second coordinate system based on the second image and designate the second position of the reference point (or the portion) of the surface as the second position of the surface in the second coordinate system. In some embodiments, the determining of the first position and the second position of the reference point (or the portion) of the surface in the second coordinate system may be performed in a similar manner as that of second characteristic information of an optical marker in the second coordinate system as described in connection with operation 506, and the descriptions of which are not repeated here.

In 906, the processing device 120 (e.g., the determination module 404) may determine, based on the first position and the second position of the surface of the object and a transform relationship between the first coordinate system and the second coordinate system, first motion information of the surface of the object in the first coordinate system.

In some embodiments, the obtaining of the transform relationship between the first coordinate system and the second coordinate system may be similar to or the same as the operation 806 of the process 800 as illustrated in FIG. 8.

In some embodiments, the processing device 120 may obtain second motion information of the surface of the object corresponding to the first image and the second image in the second coordinate system. if the first image may be acquired at a first moment, the second image may be acquired at a second moment, and the first moment is earlier than the second moment, the second motion information of the surface of the object may include a magnitude of the motion of the surface of the object from the first moment to the second moment.

In some embodiments, the processing device 120 may obtain a count of pixels between the first position and the second position. The processing device 120 may further determine a distance between the first position and the second position according to resolutions corresponding to the first position and the second position and the count of pixels between the first position and the second position. The processing device 120 may designate the distance between the first position and the second position as the magnitude of the motion of the surface of the object from the first moment to the second moment.

In some embodiments, the transform relationship between the first coordinate system and the second coordinate system may include a transform relationship between motion information denoted by the first coordinate system and motion information denoted by the second coordinate system. The processing device 120 may directly determine the first motion information of the surface of the object in the first coordinate system based on the second motion information of the surface of the object and the transform relationship between the first coordinate system and the second coordinate system.

In some embodiments, the processing device 120 may determine a third position of the point (or the portion) of the surface in the first coordinate system based on the first position of the point (or the portion) of the surface in the second coordinate system and the transform relationship between the first coordinate system and the second coordinate system. The processing device 120 may also determine a fourth position of the point (or the portion) of the surface in the first coordinate system based on the second position of the point (or the portion) of the surface in the second coordinate system and the transform relationship between the first coordinate system and the second coordinate system. The processing device 120 may also determine a distance between the third position and the fourth position in the first coordinate system. The processing device 120 may designate the distance between the third position and the fourth position in the first coordinate system as the first motion information of the surface of the object in the first coordinate system.

In some embodiments, the first motion information of the surface of the object may be used to indicate breathing information of the object. For example, the surface of the object may be a surface of the chest or the abdomen of the object, and the first motion information of the surface of the object may be used to indicate breathing motion of the object. The obtained breathing motion of the object may be used to guide an imaging device to perform a scan, such as a respiratory gating scan (e.g., a prospective respiratory gating scan, or a retrospective respiratory gating scan). In some embodiments, the first motion information of the surface of the object may be used to indicate a change of a position of a target area of the object. For example, the surface of the object may be a surface of the brain of the object, the first motion information of the surface of the object may be used to indicate a change of a position of the brain of the object. The processing device 120 may determine whether the target area of the object moves or whether a range of the moving of the target region of the object exceeds a preset range threshold based on the first motion information of the surface of the object.

In some embodiments, the image acquisition device may capture images of the surface of the object continuously or intermittently (e.g., periodically) before, during, and/or after a scan or a treatment of the subject performed by the medical device. In some embodiments, the acquisition of the images of the surface by the image acquisition device, the transmission of the captured images of the surface to the processing device 120, and the determining of the first motion information of the surface corresponding to two images may be performed substantially in real time so that substantially real-time status information (e.g., the breathing status) of the object may be determined based on the obtained first motion information of the surface corresponding to images of the surface. In some embodiments, the real-time status information of the object may be used to correct scanning data of the object acquired by the medical device to improve the accuracy of the scanning data of the object.

FIG. 10 is a flowchart illustrating an exemplary process 1000 for determining resolutions of an image acquisition device of a medical system according to some embodiments of the present disclosure. In some embodiments, the process 1000 may be implemented in the medical system 100 illustrated in FIG. 1. For example, the process 1000 may be stored in a storage (e.g., the storage device 150, the storage device 220, the storage device 390) as a form of instructions, and invoked and/or executed by the processing device 120 (e.g., the processor 210 of the computing device 200 as illustrated in FIG. 2, the CPU 340 of the mobile device 300 as illustrated in FIG. 3, and/or one or more modules as illustrated in FIG. 4). The operations of the illustrated process presented below are intended to be illustrative. In some embodiments, the process 1000 may be accomplished with one or more additional operations not described, and/or without one or more of the operations discussed. Additionally, the order in which the operations of the process 1000 as illustrated in FIG. 10 and described below is not intended to be limiting.

In 1002, the processing device 120 (e.g., the determination module 404) may obtain an image of one or more optical markers acquired by the image acquisition device, the one or more optical markers having different distances from the image acquisition device.

In some embodiments, the image acquisition device may be a device that can acquire depth image data of a subject. The depth image data may refer to image data that includes depth information of each physical point of the subject, such as a distance from each physical point to a specific point (e.g., an optical center of the image acquisition device). Exemplary image acquisition devices that can acquire depth image data of a subject may include a range sensing device, e.g., a structured light scanner, a time-of-flight (TOF) device, a stereo triangulation camera, a sheet of light triangulation device, an interferometry device, a coded aperture device, a stereo matching device, or the like, or any combination thereof. An optical marker refers to a marker that can reflect light. In some embodiments, an optical marker may include a 2D optical marker or a 3D optical marker. More descriptions for the image acquisition device and the one or more optical markers may be found elsewhere in the present disclosure (e.g., FIG. 1, FIG. 5A, and the descriptions thereof). In some embodiments, the obtaining of the image of the one or more optical markers may be performed in a similar manner as that of the multiple images each of which includes the one or more optical markers as described in connection with operation 504, and the descriptions of which are not repeated here.

In 1004, the processing device 120 (e.g., the determination module 404) may determine, based on sizes of the one or more optical markers in a first coordinate system and a transform relationship between the first coordinate system and a second coordinate system, sizes of the one or more optical markers in the image.

In some embodiments, the first coordinate system and the second coordinate system may be the same as or similar to the first coordinate system and the second coordinate system described in FIG. 5A. In some embodiments, the obtaining of the transform relationship between the first coordinate system and the second coordinate system may be similar to or the same as the operation 806 of the process 800 as illustrated in FIG. 8.

In some embodiments, for each of the one or more optical markers, the processing device 120 may obtain a size of the optical marker in the first coordinate system. The processing device 120 may determine a size of the optical marker in the second coordinate system according to the size of the optical marker in the first coordinate system and the transform relationship between the first coordinate system and a second coordinate system.

In 1006, the processing device 120 (e.g., the determination module 404) may determine, based on the sizes of the one or more optical markers in the image, resolutions of the image acquisition device for different distances from the image acquisition device.

In some embodiments, for each of the one or more optical markers, the processing device 120 may obtain a count of pixels in the image corresponding to the optical marker. The processing device 120 may determine a resolution of the image acquisition device corresponding to a distance between the optical marker and the image acquisition device based on the size of the optical marker in the image and the count of pixels in the image corresponding to the optical marker. For example, the processing device 120 may designate a value of the size of the optical marker in the image divided by the count of pixels in the image corresponding to the optical marker as the resolution of the image acquisition device corresponding to the distance between the optical marker and the image acquisition device.

According to some embodiments of the present disclosure, the resolutions of the image acquisition device for different distances from the image acquisition device may be determined automatically based on the one or more optical markers and the transform relationship between the first coordinate system and the second coordinate system, which is simple and has a high efficiency and accuracy.

It will be apparent to those skilled in the art that various changes and modifications can be made in the present disclosure without departing from the spirit and scope of the disclosure. In this manner, the present disclosure may be intended to include such modifications and variations if the modifications and variations of the present disclosure are within the scope of the appended claims and the equivalents thereof.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

1. A system, comprising:
at least one storage device including a set of instructions; and
at least one processor in communication with the at least one storage device, wherein when executing the set of instructions, the at least one processor is directed to cause the system to perform operations including:
obtaining first characteristic information of one or more optical markers in a first coordinate system applied to a medical device;
obtaining multiple images each of which includes the one or more optical markers acquired by an image acquisition device, wherein a field of view of the image acquisition device covers a scan region of the medical device;
obtaining, based on the multiple images, second characteristic information of the one or more optical markers in a second coordinate system applied to the multiple images acquired by the image acquisition device; and
determining, based on the first characteristic information and the second characteristic information of the one or more optical markers, a transform relationship between the first coordinate system and the second coordinate system.

2. The system of claim 1, wherein the one or more optical markers are disposed on at least one of a couch of the medical device or an inner wall of a bore of the medical device.

3. The system of claim 1, at least one of the one or more optical markers is disposed on an end of a couch of the medical device near a bore of the medical device.

4. The system of claim 1, wherein an optical marker among the one or more optical markers is disposed on a couch of the medical device, the first characteristic information of the optical marker includes first position information of the optical marker, and obtaining the first characteristic information of the optical marker in the first coordinate system applied to the medical device includes:
obtaining positions of the couch in the first coordinate system during movement of the couch; and
determining, based on the positions of the couch in the first coordinate system, the first position information of the optical marker in the first coordinate system.

5. The system of claim 1, wherein the at least one processor is further directed to cause the system to perform operations including:
obtaining multiple groups of status parameters of a couch of the medical device, each group including a load and a first extension of the couch along a longitudinal direction of the couch;
for each group of the multiple groups of status parameters of the couch,
obtaining a first image of the couch including an optical marker disposed on the couch acquired by the image acquisition device when the couch is under the load and the first extension in the group;
determining, based on the first image of the couch, a first position of the optical marker in the second coordinate system;
determining, based on the first position of the optical marker and a prior transform relationship between the first coordinate system and the second coordinate system, a first deformation level of the couch in the first coordinate system; and
performing, based on the multiple groups of the status parameters of the couch and first deformation levels corresponding to the multiple groups of the status parameters of the couch, a calibration on one or more performance parameters of the couch to obtain a calibration result.

6. The system of claim 1, wherein the at least one processor is further directed to cause the system to perform operations including:
obtaining a weight of an object and a second extension of a couch of the medical device after the couch moves the object to a position in a bore of the medical device;
obtaining a second image of the couch including the optical marker disposed on the couch acquired by the image acquisition device after the couch moves the object to the position in the bore of the medical device;
determining, based on the second image, a second position of the optical marker in the second coordinate system;
determining, based on the second position of the optical marker and the transform relationship between the first coordinate system and the second coordinate system, a second deformation level of the couch in the first coordinate system; and
determining, based on the weight of the object, the second extension, the second deformation level of the couch, and a calibration result corresponding to one or more performance parameters of the couch, an assessment result of the couch.

7. The system of claim 1, wherein the at least one processor is further directed to cause the system to perform operations including:
obtaining a first image and a second image each of which includes a surface of an object being scanned acquired by the image acquisition device;
obtaining a first position of the surface in the second coordinate system based on the first image and a second position of the surface in the second coordinate system based on the second image; and
determining, based on the first position and the second position of the surface of the object and the transform relationship between the first coordinate system and the second coordinate system, motion information of the surface of the object in the first coordinate system.

8. The system of claim 1, wherein, the at least one processor is further directed to cause the system to perform operations including:
obtaining an image of the one or more optical markers acquired by the image acquisition device, the one or more optical markers having different distances from the image acquisition device; and
determining, based on sizes of the one or more optical markers in the first coordinate system, and the transform relationship, sizes of the one or more optical markers in the image; and
determining, based on the sizes of the one or more optical markers in the image, resolutions of the image acquisition device for different distances from the image acquisition device.

9. A system, comprising:
at least one storage device including a set of instructions; and
at least one processor in communication with the at least one storage device, wherein when executing the set of instructions, the at least one processor is directed to cause the system to perform operations including:
obtaining a weight of an object and an extension of a couch of a medical device along a longitudinal direction of the couch after the couch moves the object to a position in a bore of the medical device;
obtaining an image of the couch including an optical marker disposed on the couch acquired by an image acquisition device after the couch moves the object to the position in the bore of the medical device, wherein a field of view of the image acquisition device covers a scan region of the medical device;
obtaining a transform relationship between a first coordinate system applied to the medical device and a second coordinate system applied to the image acquisition device;
determining, based on the image of the couch, a position of the optical marker in the second coordinate system;
determining, based on the position of the optical marker and the transform relationship between the first coordinate system and the second coordinate system, a deformation level of the couch in the first coordinate system; and
determining, based on the weight of the object, the extension, the deformation level of the couch, and a calibration result corresponding to one or more performance parameters of the couch, an assessment result of the couch.

10. The system of claim 9, wherein obtaining the calibration result of the couch comprises:
obtaining multiple groups of status parameters of the couch, each group including a load and a second extension of the couch along the longitudinal direction of the couch;
for each group of the multiple groups of status parameters of the couch;
obtaining a second image of the couch including an optical marker disposed on the couch acquired by the image acquisition device when the couch is under the load and the second extension in the each group;
determining, based on the second image of the couch, a second position of the optical marker in the second coordinate system;
determining, based on the second position of the optical marker and a prior transform relationship between the first coordinate system and the second coordinate system, a second deformation level of the couch in the first coordinate system; and
performing, based on the multiple groups of the status parameters of the couch and second deformation levels corresponding to the multiple groups of the status parameters of the couch, a calibration on one or more performance parameters of the couch to obtain the calibration result.

11. The system of claim 9, wherein the obtaining the transform relationship between a first coordinate system applied to the medical system and a second coordinate system applied to the image acquisition device comprises:
obtaining first characteristic information of one or more optical markers in the first coordinate system;
obtaining multiple third images each of which includes the one or more optical markers acquired by the image acquisition device;
obtaining, based on the multiple third images, second characteristic information of the one or more optical markers in the second coordinate system; and
determining, based on the first characteristic information and the second characteristic information of the one or more optical markers, the transform relationship between the first coordinate system and the second coordinate system.

12. The system of claim 11, wherein the one or more optical markers are disposed on at least one of a couch of the medical device or an inner wall of a bore of the medical device.

13. The system of claim 11, at least one of the one or more optical markers is disposed on an end of a couch of the medical device near a bore of the medical device.

14. The system of claim 11, wherein an optical marker among the one or more optical markers is disposed on a couch of the medical device, the first characteristic information of the optical marker includes first position information of the optical marker, and obtaining the first characteristic information of the optical marker in the first coordinate system applied to the medical device includes:
obtaining positions of the couch in the first coordinate system during movement of the couch; and
determining, based on the positions of the couch in the first coordinate system, the first position information of the optical marker in the first coordinate system.

15. The system of claim 11, wherein the at least one processor is further directed to cause the system to perform operations including:
obtaining a first image and a second image each of which includes a surface of an object being scanned acquired by the image acquisition device;
obtaining a first position of the surface in the second coordinate system based on the first image and a second position of the surface in the second coordinate system based on the second image; and
determining, based on the first position and the second position of the surface of the object and the transform relationship between the first coordinate system and the second coordinate system, motion information of the surface of the object in the first coordinate system.

16. The system of claim 11, wherein, the at least one processor is further directed to cause the system to perform operations including:
obtaining an image of the one or more optical markers acquired by the image acquisition device, the one or more optical markers having different distances from the image acquisition device; and
determining, based on sizes of the one or more optical markers in the first coordinate system, and the transform relationship, sizes of the one or more optical markers in the image; and
determining, based on the sizes of the one or more optical markers in the image, resolutions of the image acquisition device for different distances from the image acquisition device.

17. A method, the method being implemented on a computing device having at least one storage device and at least one processor, the method comprising:
obtaining first characteristic information of one or more optical markers in a first coordinate system applied to a medical device;
obtaining multiple images each of which includes the one or more optical markers acquired by an image acquisition device, wherein a field of view of the image acquisition device covers a scan region of the medical device;
obtaining, based on the multiple images, second characteristic information of the one or more optical markers in a second coordinate system applied to the multiple images acquired by the image acquisition device; and
determining, based on the first characteristic information and the second characteristic information of the one or more optical markers, a transform relationship between the first coordinate system and the second coordinate system.

18-20. (canceled)

21. The method of claim 17, wherein the one or more optical markers are disposed on at least one of a couch of the medical device or an inner wall of a bore of the medical device.

22. The method of claim 17, wherein at least one of the one or more optical markers is disposed on an end of a couch of the medical device near a bore of the medical device.

23. The method of claim 17, wherein an optical marker among the one or more optical markers is disposed on a couch of the medical device, the first characteristic information of the optical marker includes first position information of the optical marker, and obtaining the first characteristic information of the optical marker in the first coordinate system applied to the medical device includes:
obtaining positions of the couch in the first coordinate system during movement of the couch; and
determining, based on the positions of the couch in the first coordinate system, the first position information of the optical marker in the first coordinate system.
